(19) United States
(12) Patent Application Publication
(10) Pub. No.: US 2024/0326335 A1
(43) Pub. Date: Oct. 3, 2024

(54) SYSTEM AND METHOD FOR THE FABRICATION OF ALIGNED STRUCTURES BY OPTICAL MODULATION INSTABILITY

(71) Applicant: Readily3D SA, Lausanne (CH)

(72) Inventors: Paul DELROT, Morges (CH); Emma BARBIER, Ecully (CH); Damien LOTERIE, Ecublens (CH)

(73) Assignee: Readily3D SA, Lausanne (CH)

(21) Appl. No.: 18/575,003

(22) PCT Filed: Jun. 27, 2022
(86) PCT No.: PCT/EP2022/067545

§ 371 Date: Dec. 28, 2023

(30) Foreign Application Priority Data
Jul. 1, 2021 (WO) PCT/EP2021/068260

(51) Int. Cl. B29C 64/277; B29C 64/118; B29C 64/124; B29C 64/30; B29K 105/00; B29K 105/16; B33Y 10/00; B33Y 30/00; B33Y 40/20

(52) U.S. Cl. CPC B29C 64/277; B29C 64/118; B29C 64/124; B29C 64/30; B33Y 10/00; B33Y 30/00; B33Y 40/20; B29K 2105/0061; B29K 2105/16

(57) ABSTRACT

A method and system for fabricating a structure (32) having at least one dimension and made of highly aligned structural micro-filaments (11), the method comprising: a) providing a photoresponsive material (13), said photoresponsive material (13) being capable of altering its material phase upon illumination by light (15) of one or more wavelengths, in an extrusion unit (12), preferably a nozzle, or in an optically transparent vessel; b) irradiating said photoresponsive material (13), preferably concurrently with its extrusion through said extrusion unit (12), with one or more light sources (14, 21, 50, 62, 65) capable of emitting light of one or more wavelengths, at least one of said one or more light sources (14) having the capability to generate an optical modulation instability in said photoresponsive material (13), thereby creating an optical modulation instability in said photoresponsive material (13), thereby forming a micro-patterned filament (10); c) forming from said formed micro-patterned filament, preferably by deposition or sequential curing, said structure having at least one dimension and made of highly aligned structural micro-filaments.

### BACKGROUND OF THE INVENTION

### 1. Field of the Invention

The present invention relates to a method and apparatus to fabricate one-dimensional, two-dimensional or three-dimensional objects made of highly aligned micro-filaments by altering the material phase of a photoresponsive material with a light source using photopolymerization-induced optical modulation instability. In particular, the present invention is related to biofabrication applications wherein the biofabricated objects locally require highly aligned structures to guide cellular growth or function.

### 2. Background Art

In tissue engineering or regenerative medicine, producing two- or three-dimensional objects s with locally aligned micro-architectures is of paramount importance to recapitulate biological function. Recapitulating biological function would benefit to drug development and to the whole pharmaceutical industry by creating functional tissues or organ models. In addition, the growing field of cellulose nanofibrils, which constitutes one of the building blocks for high-performance biomaterials and textiles, would also benefit from an industrially applicable tool for the production of highly-aligned micro-structures.

State-of-the-art methods and systems to create aligned nano- and micro-filaments are electrospinning, melt electro-writing and micro-extrusion. Though electrospinning and melt electro-writing can be used for direct-writing of 2D and 3D woven structures made of fibers of diameters ranging from <100 nm to 500 μm, these techniques are not suitable for bio-fabrication since they use solvents and high voltages that are detrimental to cell viability (Toby D. Brown, Paul D. Dalton, Dietmar W. Hutmacher, Melt electrospinning today: An opportune time for an emerging polymer process, Progress in Polymer Science, Volume 56, 2016). These techniques are also hardly scalable. With current fiber production rates in the order of meters per minute, they have little industrial applicability and are limited to research purposes.

Micro-extrusion has been employed in different embodiments (WO 2015/017421 A2, US-2016/0288414 A1, U.S. Pat. No. 9,433,969 B2, EP-2 969 488 B1) to fabricate two-dimensional and three-dimensional objects made of micrometric fibers. Extrusion through a nozzle results in shear stresses onto the extruded material, which degrades cell viability. For the same reason, seeding cells into extruded fibers of diameters within the same size range as cells (5-20 μm) is not practical. Micro-structuration of cell-seeded objects at a scale essential to provide effective cell guidance is thus limited. Though recent work on microcompartmentalization (Samandari et al., Controlling cellular organization in bioprinting through designed 3D microcompartmentalization, Applied Physics Reviews 8, 021404 (2021)) demonstrated the possibility to extrude filaments composed of highly-aligned micro-fibrils, this embodiment does not offer a means to dynamically control the geometry and repartition of such micro-fibrils.

Optical modulation instability is characterized by the spontaneous break-up of a uniform light beam into smaller light beams (Biria et al., Coupling nonlinear optical waves to photoreactive and phase-separating soft matter: current status and perspectives, Chaos 27, 104611 (2017)). This phenomenon occurs in materials exhibiting a non-linear response, wherein the noise of the optical light beam is locally amplified by the material's non-linearity. When a light beam carrying noise propagates in a self-focusing medium, it experiences slightly higher refractive index in regions with slightly higher intensity. As the light propagates, the higher refractive index regions attract more light nearby and yield even higher refractive indices that attract even more light. If this self-focusing effect through positive feedback is stronger than the diffraction effect for the light beam, the light begins to localize. This more localized light then causes the diffraction effect to grow. When finally the diffraction is sufficient to balance the self-focusing, the geometry of the modulation instability is determined and modulation instability patterns form.

Photoresponsive materials such as photopolymer resins inherently have an integrated non-linear behavior since their refractive index increases during curing by light. Hence, when exposed with a uniform and partially coherent light beam, a photopolymer will cure faster in local areas of higher intensity, thus locally creating cores of higher refractive index which result in optical self-trapping and the formation of highly-aligned micrometric filaments.

Spatial masks were used in the various embodiments of micro-fabrication (U.S. Pat. No. 8,435,438 B1; Kim, One-Step 3D Microfabrication of High-Resolution, High-Aspect-Ratio Micropillar Arrays for Soft Artificial Axons by Using Light-Induced Self-Focusing Photopolymerization, J. Korean Soc. Precis. Eng., 36, 4, 425-429), using optical modulation instability in photoresponsive materials in order to circumvent the need of a partially spatially coherent light beam.

Though optical modulation instability has the potential to produce micro-structured objects, it has so far found limited applications because the size of the produced structures is limited to the millimeter range and simple structures (Ponte et al., Self-Organized Lattices of Nonlinear Optochemical Waves in Photopolymerizable Fluids: The Spontaneous Emergence of 3-D Order in a Weakly Correlated System, J. Phys. Chem. Lett., 2018, 9, 1146-1155).

As a consequence, there is a need for an industrially applicable system to produce objects with locally highly-aligned micro-architectures.

### SUMMARY OF THE INVENTION

The present invention circumvents all of the previous shortcomings of methods and systems for the production of objects with locally highly-aligned micro-architectures.

The invention herein disclosed provides a method and light-based system to produce one-dimensional, two-dimensional or three-dimensional objects made of highly aligned photopolymer micro-filaments of arbitrary length with a tunable diameter, a tunable crosslinking degree, and which are assembled in a bundle of tunable shape.

Accordingly, the present invention provides a method for fabricating a structure having at least one dimension and made of highly aligned structural micro-filaments, the method comprising:
a) providing a photoresponsive material, said photoresponsive material being capable of altering its material phase upon illumination by light of one or more wavelengths, in an extrusion unit, preferably a nozzle, or in an optically transparent vessel;
b) irradiating said photoresponsive material, preferably concurrently with its extrusion through said extrusion unit, with one or more light sources capable of emitting light of one or more wavelengths, at least one of said one or more light sources having the capability to generate an optical modulation instability in said photoresponsive material, thereby creating an optical modulation instability in said photoresponsive material, thereby forming a micro-patterned filament;
c) forming from said formed micro-patterned filament, preferably by deposition or sequential curing, said structure having at least one dimension and made of highly aligned structural micro-filaments.

In a preferred embodiment, the method comprises the following steps:
a) Providing a photoresponsive material capable of altering its material phase upon illumination by light with one or more wavelengths;
b) Providing one or more projections units capable of emitting spatial patterns of light with one or more wavelengths, at least one of said projection units of spatial light patterns having the capability to generate an optical modulation instability in said photoresponsive material;
c) Providing an extrusion unit, preferably a nozzle, to deposit said photoresponsive material;
d) Optionally providing a support material for embedded printing of said structure, wherein said support material is a Bingham plastic material;
e) Computing a sequence of projections for at least one of said one or more projection units, said sequence of projections describing the micro-structural components of said structure;
f) Defining a sequence of light patterns using said sequences of projections;
g) Extruding said photoresponsive material through said extrusion unit, preferably said nozzle, and concurrently irradiating said photoresponsive material with said sequences of light patterns from said one or more projection units, thereby creating a patterned optical modulation instability in said extruded photoresponsive material, thereby forming a micro-patterned filament;
h) Concurrently depositing the formed micro-patterned filament to form the fabricated structure, optionally performing said deposition in said support material, thereby fabricating said structure made of highly aligned structural micro-components;
i) Optionally removing said support material to release said fabricated structure from said support material.

In a preferred embodiment, the method of the present invention comprises the following further steps for producing a multi-material structure:
Providing another photoresponsive material capable of altering its material phase upon illumination by one or more wavelengths of light;
Repeating steps (a) to (b) with said other photoresponsive material in the method described above until said multi-material structure is produced.

According to a further embodiment, the present invention provides another method for fabricating a structure having at least one dimension and having highly aligned structural micro-components, the method comprising:
a) Providing an optically transparent vessel containing a photoresponsive material capable of altering its material phase upon illumination by one or more wavelengths of light;
b) Providing one or more, preferably at least two, projections units capable of emitting spatial patterns of light with one or more wavelengths, at least one of said one or more projection units of spatial light patterns having the capability to generate an optical modulation instability in said photoresponsive material;
c) Computing a sequence of projections for at least one, preferably each, of said one or more projection units describing the different layers of the structure to be fabricated;
d) Defining a sequence of light patterns for at least one, each of said one or more projection units using said computed sequence of projections;

e) Irradiating said photoresponsive material with said light patterns according to the defined sequences, thereby sequentially generating a layer of the structure with an optical modulation instability, thereby sequentially curing a layer of said structure, said layer being composed of highly aligned micro-filaments generated by said optical modulation instability, thereby fabricating said structure made of highly aligned structural micro-filaments.

In a further preferred embodiment, the above method comprises the following further steps for producing a multi-material structure:
Removing uncured parts of said photoresponsive material and immersing said fabricated structure into another photoresponsive material into said optical vessel;
Repeating the steps (c) to (f) of the above method until said multi-material structure is produced.

In a further preferred embodiment of said method of the present invention, the relative position of said optical vessel and at least one of said one or more projection units can be actuated and controlled concurrently to said irradiation of said optical vessel.

According to a further embodiment, the present invention provides another method for fabricating a structure having at least one dimension and having highly aligned structural micro-components, the method comprising:
a) Providing an optically transparent vessel, preferably a transparent mold, containing a photoresponsive material capable of altering its material phase upon illumination by one or more wavelengths of light;
b) Providing one or more, preferably at least two, light sources capable of emitting light with one or more wavelengths, at least one of said one or more light sources having the capability to generate an optical modulation instability in said photoresponsive material;
c) irradiating said photoresponsive material with said one or more light sources capable of emitting light of one or more wavelengths, at least one of said one or more light sources having the capability to generate an optical modulation instability in said photoresponsive material, thereby creating an optical modulation instability in said photoresponsive material, thereby forming a molded structure made of highly-aligned structural micro-components;
d) Optionally, removing said structure from said optically transparent vessel.

In a further preferred embodiment, the above method comprises the following further steps for producing a multi-material structure:
Removing uncured parts of said photoresponsive material and immersing said fabricated structure into another photoresponsive material into said optical vessel;
Repeating the step (c) of the above method until said multi-material structure is produced.

In a further preferred embodiment of said method of the present invention, the relative position of said optical vessel and at least one of said one or more projection units can be actuated and controlled concurrently to said irradiation of said optical vessel.

In a further embodiment of the present invention, the spatial coherence of at least one of said one or more light sources or said one or more projection units can be controlled and actuated concurrently to said irradiation, thereby modifying the size of said micro-patterns on said formed filament.

Moreover, the present invention provides a system for the fabrication of a structure having at least one dimension and made of highly aligned structural micro-components, the system comprising:
a) A unit in which a photoresponsive material can be irradiated, said unit being selected from the group consisting of an extrusion unit, preferably a nozzle, and an optically transparent vessel;
b) One or more light sources, preferably projection units, capable of emitting light of one or more wavelengths into said unit, wherein at least one of said one or more light sources is configured to emit light that is capable of generating an optical modulation instability in said photoresponsive material;
c) A means for controlling said one or more light sources, said means being configured to cause emission of light from at least one of said light sources that is capable of generating an optical modulation instability in said photoresponsive material;
d) optionally, a means for controlling an extrusion of said photoresponsive material through said extrusion unit, preferably nozzle;
e) optionally, a support (30, 41), said support (30, 41) being selected from the group consisting of a print bed (30) and a container (41);
f) optionally, a means for controlling the relative spatial position of said extrusion unit, preferably nozzle and said support (30, 41);
g) optionally, a means for actuating and controlling the relative spatial position of said optically transparent vessel and said one or more light sources, preferably projection units.
h) Optionally, a means for controlling the spatial coherence of said one or more light sources;
i) Optionally, a means for guiding light from said one or more light sources towards said extrusion unit, preferably nozzle.

In a further embodiment of the present invention, said one or more projection units comprises a light source capable of emitting one or more wavelengths of light and at least one of a spatial light modulator, a digital micromirror device, a galvanometer-scanner, an acousto-optic deflector, a lens, a multimode fiber or a bundle of multimode fibers.

In a further embodiment of the present invention, at least one of said one or more light sources is spatially coherent with a beam-parameter product less than 400 μm·rad, preferably less than 100 μm·rad, most preferably less than 50 μm·rad.

In a further embodiment of the invention, at least one of said one or more projection units is configured to irradiate said optically transparent vessel with a light sheet.

In a further embodiment of the invention, said photoresponsive material is seeded with cells.

### BRIEF DESCRIPTION OF THE DRAWINGS

These and other features, aspects and advantages of the present invention will become better understood with regard to the following non-limiting description and drawings where:

FIG. 1: is a schematic illustration of a first embodiment of a system of the present invention for producing highly-aligned micro-filaments.

FIG. 2: is a schematic illustration of one alternative variant of the system of FIG. 1.

FIG. 3: is a schematic illustration of another alternative variant of the system of FIG. 1.

FIG. 4: is a schematic illustration of another alternative variant of the system of FIG. 1.

FIG. 5: is a schematic illustration of another embodiment of the present invention wherein a structure is fabricated with highly-aligned structural micro-components of tunable geometry.

FIG. 6: is a schematic illustration of another embodiment of the present invention wherein a structure is fabricated with highly-aligned structural micro-components, and a dual-color photoresponsive material is used.

FIG. 6A: is a schematic illustration of another embodiment of the present invention wherein a structure is fabricated with highly-aligned structural micro-components.

FIG. 6B: is a schematic illustration of another embodiment of the present invention wherein a structure is fabricated with highly-aligned structural micro-components according to various directions using a transparent mold.

FIG. 7: is a schematic illustration of an alternative embodiment of the present invention wherein a multimode optical fiber is used to guide light towards a nozzle.

FIG. 8: is a schematic illustration of an alternative embodiment of the present invention wherein a microfluidic chip is used.

FIG. 9: is a schematic illustration of an alternative embodiment of the present invention wherein a microfluidic chip is used with a sacrificial material.

FIG. 10: is a picture of a device according to one embodiment of the present invention wherein a microfluidic chip is used.

FIG. 11: is another picture of the device shown in FIG. 10.

FIG. 12: is a computer-rendered image of a microfluidic chip with a sacrificial material used in the alternative embodiment described in FIG. 9.

FIG. 13: is a picture of a device according to one embodiment of the present invention wherein a multimode optical fiber is used.

FIG. 14: is a picture of a filament with highly-aligned micro-filaments obtained with the device according to the embodiment depicted in FIG. 10.

FIG. 15: is a picture of a filament with highly-aligned micro-filaments obtained with the device according to the embodiment depicted in FIG. 13.

FIG. 16: is a schematic illustration of another alternative variant of the system of FIG. 1.

FIG. 17: is a schematic illustration of another alternative variant of the system of FIG. 1.

In the figures, same reference numbers denote the same components.

### DETAILED DESCRIPTION

According to the present invention, the term “altering its material phase” indicates that a material, preferably a photoresponsive material, may undergo a phase transition, preferably from the liquid to the solid state or to a gel state, or from the gel to the solid state.

According to the present invention, the term “light source having the capability to generate an optical modulation instability in said photoresponsive material” defines that the respective light source can emit light that is capable of generating, in a photoresponsive material, an optical modulation instability. This means that the light source can emit light that is at least partially spatially coherent, wherein the spatial coherence of a light source can be defined by the beam-parameter product of the source, that is to say, the product of its emitting size by its divergence. Partially spatially coherent means that the beam-parameter product of the source is preferably less than 400 μm·rad, preferably less than 100 μm·rad, preferably less than 50 μm·rad.

According to the present invention, the term “additive manufacturing” refers to methods where a volume of photoresponsive material is irradiated, in order to fabricate a three-dimensional object. Examples of additive manufacturing methods that can be used according to the present invention are xolography and tomographic additive manufacturing. An apparatus for tomographic additive manufacturing is described in detail in e.g. WO 2019/043529 Al or US 2018/0326666 A1.

According to the present invention, the term “micro-filaments” and “micro-components” are used interchangeably.

The present invention is related to light-based additive manufacturing methods and systems.

The present invention provides a way for end-users of additive manufacturing machines to produce structures made of locally highly-aligned micro-filaments with tunable parameters, including the micro-filaments' shape, size and geometry. The flexible and versatile tools and methods provided in the present invention circumvent the shortcomings of state-of-the-art additive manufacturing devices, thus leading to a wider range of research and industrial applications of additive manufacturing.

In additive manufacturing, and more specifically in biofabrication, producing structures with locally highly aligned micro-architectures, a scale relevant to growth guidance of cells, is critical to create functional tissue and organ models. Creating functional tissue and organ models would facilitate and reduce the cost of drug development by allowing for drug screening on models reproducing with more fidelity the behavior or native tissues and organs. In addition, the production of functional tissue and organ models would reduce the use of animal models.

According to the present invention, it was found that generating an optical modulation instability in a photoresponsive material can be used for producing highly-aligned micro-filaments of arbitrary length, shape and size. Assemblies of these micro-filaments can further be made to create two-dimensional or three-dimensional structures.

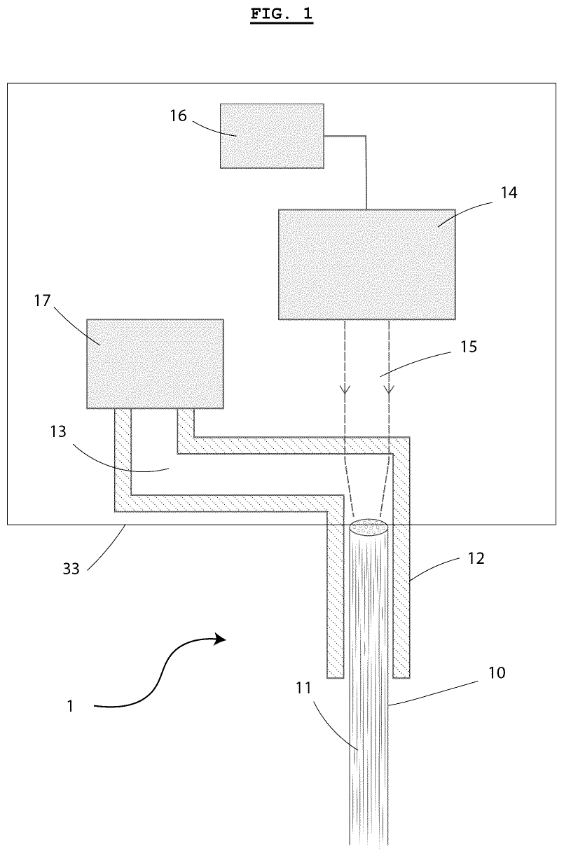

An embodiment of the present invention is described in FIG. 1 and provides for a system 1 for producing highly-aligned micro-filaments. In detail, the system 1 according to this embodiment for the fabrication of an arbitrary length of a filament 10 made of highly aligned micro-filaments 11 comprises:
An extrusion unit, preferably a nozzle, 12 to extrude a photoresponsive material 13;
A print head 33 comprising a light source 14 capable of emitting one or more wavelengths of light 15, wherein the light source 14 is capable of generating an optical modulation instability in the photoresponsive material 13; wherein the light source 14 is arranged in the system such that it can irradiate the photoresponsive material 13 when being in the extrusion unit 12;
and further comprising a means 16 for controlling the intensity of the light source 14, said means being configured to cause emission of light from said light sources 14 that is capable of generating an optical modulation instability in said photoresponsive material;
a means 17 for controlling the extrusion of the photoresponsive material 13 through the extrusion unit, preferably nozzle 12.

Said light source 14 may be any known light source that is capable of emitting partially coherent light. The light source can be, but is not limited to, a laser diode, a LED, an OLED, a diode-pumped solid-state laser, an incandescent filament and a combination of multiple LEDs.

Said means 16 for controlling the intensity of the light source 14 can be any known means that is conventionally used for this purpose, and is configured to cause emission of light from said light sources that is capable of generating an optical modulation instability in said photoresponsive material. For example, a conventional processing unit such as a computer may be used, which is respectively configured, for example, by the provision of a respective software.

Said means 17 for controlling the extrusion of the photoresponsive material may be any known means that is conventionally used for this purpose. Said means 17 is connected to a container such as a tank in which said photoresponsive material is stored, and controls the transport of said photoresponsive material. Said means 17 may be controlled, for example, by a conventional processing unit such as a computer. Said means 17 may further comprise a locking unit such as a valve or a flap.

In a preferred embodiment, said means 17 for controlling the extrusion of said photoresponsive material is selected from the group consisting of a pump, a syringe pump or a peristaltic pump.

In another embodiment, said means 17 for controlling the extrusion of said photoresponsive material provides a way of controlling the material flow rate, by varying either the dispensing nozzle cross-sectional area and/or the material flow velocity. This can be achieved, for example, by the provision of a locking unit such as a valve, or by regulating the operation of said means 17 being selected from the group consisting of a pump, a syringe pump or a peristaltic pump.

In a further embodiment, said means 17 for controlling said extrusion allows for a co-linear and patterned flow of two or more different materials, thus allowing the implementation of multi-material three dimensional constructs. Patterned flow here means that the cross-section of the co-linear flow of materials keeps a defined spatial pattern as they flow, because they do not mix.

In a further embodiment, said means 17 for controlling said extrusion allows for heating or cooling of the photoresponsive material to be dispensed. This can be achieved, for example, by providing conventional heating or cooling units in said means 17.

In a further embodiment, said means 17 for controlling said extrusion allows for loading of compatible material cartridges, which can be sterilized. This can be achieved, for example, by providing a cartridge made of an autoclavable container and a Luer-lock outlet that can be fitted to a Luer-lock inlet of said means for controlling said extrusion.

In a further embodiment, said cartridges can be kept at a controlled temperature.

In a preferred embodiment, the system further comprises an oxygen diffusing unit at one or more specific sites of the system, thus providing an additional control over the photoresponsive material response to light, since oxygen radical scavenging inhibits polymerization.

Said extrusion unit 12 may be any conventional unit through which a material may be extrude. Preferably, said extrusion unit is a nozzle.

According to this embodiment, in said system 1 there can be carried out a method of forming a filament 10 of an arbitrary length made of highly aligned micro-filaments 11, wherein the method comprises the steps of:
a) extruding a photoresponsive material 13, said photoresponsive material 13 being capable of altering its material phase upon illumination by light of one or more wavelengths, through an extrusion unit 12, preferably a nozzle;
b) concurrently with said extrusion, irradiating said photoresponsive material 13 with one or more light sources 14 capable of emitting light 15 of one or more wavelengths, at least one of said one or more light sources 14 having the capability to generate an optical modulation instability in said photoresponsive material 13, thereby creating an optical modulation instability in said extruded photoresponsive material 13, thereby forming a micro-patterned filament 10;
c) depositing said formed micro-patterned filament 10 to form said structure having at least one dimension and made of highly aligned structural micro-filaments 11.

Step a) is performed by transporting said photoresponsive material 13 into said extrusion unit 12, with the aid of said means 17. In said extrusion unit 12, preferably a nozzle, the photoresponsive material 13 is irradiated with light 15 emitted from said light source 14. The light source 14 is arranged in the print head 33 such that its emitted light may enter into the extrusion unit 12, preferably nozzle. As explained above, said light has to be capable of generating an optical modulation instability in said photoresponsive material 13. If such light 15 is irradiated into said photoresponsive material 13, by the mechanism of optical modulation instability described above it will evoke the formation of micro-filaments 11 in said photoresponsive material 13. As a result, a micro-patterned filament 10 made of highly aligned structural micro-filaments 11 is formed in said extrusion unit 12 and deposited by exiting from said extrusion unit 12.

According to this embodiment, in said system 1 there can be carried out a method of forming a filament 10 of an arbitrary length made of highly aligned micro-filaments 11, wherein the method comprises the steps of:
a) extruding a photoresponsive material 13, said photoresponsive material 13 being capable of altering its material phase upon illumination by light of one or more wavelengths, through an extrusion unit 12, preferably a nozzle;
b) after said photoresponsive material 13 has been extruded, irradiating said photoresponsive material 13 with one or more light sources 14 capable of emitting light 15 of one or more wavelengths, at least one of said one or more light sources 14 having the capability to generate an optical modulation instability in said photoresponsive material 13, thereby creating an optical modulation instability in said extruded photoresponsive material 13, thereby forming a micro-patterned filament 10;
c) forming said structure having at least one dimension and made of highly aligned structural micro-filaments 11 using said formed micro-patterned filament 10.

Step a) is performed by transporting said photoresponsive material 13 into said extrusion unit 12, with the aid of said means 17. The light source 14 is arranged in the print head 33 such that its emitted light may irradiate said extruded photoresponsive material after it exited said extrusion unit 12, preferably nozzle. As explained above, said light has to be capable of generating an optical modulation instability in said photoresponsive material 13. If such light 15 is irradiated into said photoresponsive material 13, by the mechanism of optical modulation instability described above it will evoke the formation of micro-filaments 11 in said photoresponsive material 13. As a result, a micro-patterned filament 10 made of highly aligned structural micro-filaments 11 is formed.

In another embodiment of the present invention, the system 1 comprises two or more light sources 14, 21 capable of emitting one or more wavelengths of light, wherein at least one of said one or more light sources 14, 21 is capable of generating an optical modulation instability in said photoresponsive material 13, as described above.

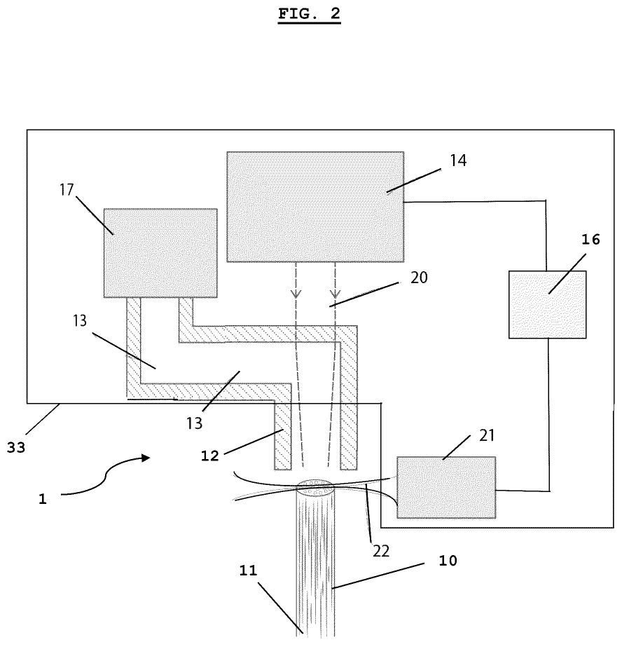

A non-limiting example of said variant with multiple light sources is shown in FIG. 2. A first light source 14 emitting light at a first wavelength 20 is arranged to irradiate the photoresponsive material 13 when being in the extrusion unit 12, preferably nozzle. This first light source 14 is capable of generating an optical modulation instability in the photoresponsive material 13. The second light source 21 emitting at a second wavelength of light 22 is arranged to irradiate the photoresponsive material 13 orthogonally to the extrusion direction, as defined by the axis of the extrusion unit, preferably nozzle 12. Both light sources 14, 21 may be controlled by the same means 16 as described above with respect to the embodiment of FIG. 1. However, it is also possible to control each of the light sources 14, 21 separately with a separate means for controlling the intensity of the light source.

It is particularly advantageous to cure the photoresponsive material just after the extrusion unit 12, preferably nozzle, to prevent any clogging of the system by the formed micro-patterned filament 10.

The embodiment described in FIG. 2 is of particular interest when using a photoresponsive material 13 that changes its material phase only upon the concurrent irradiation by two different wavelengths of light and does not change its material phase when irradiated by only one of the first or second wavelength of light.

Such photoresponsive materials are known, for example from EP-3 691 860 A1. It may be, for example, a liquid photoresponsive material that comprises a first photoinitiator and a second photoinitiator, wherein said first and second photoinitiator interact with light of different wavelengths. Alternatively, the photoresponsive material may comprise a photoinhibitor that interacts with a second wavelength of light to selectively hinder the ability of a first wavelength of light to alter the phase of said photoresponsive material.

According to another alternative embodiment, the photoresponsive material may comprise a two-stage photo-initiator, such that said photoresponsive material is locally altered upon local simultaneous or successive illumination with first and second wavelengths of light but not altered if locally illuminated with only one of the wavelengths of light. Such two-stage photoinitiators are known and described, for example, in Regehly et al., Nature, Vol. 588 (2020), 620-624. An example is a spiropyran as described in Regehly et al., ibid).

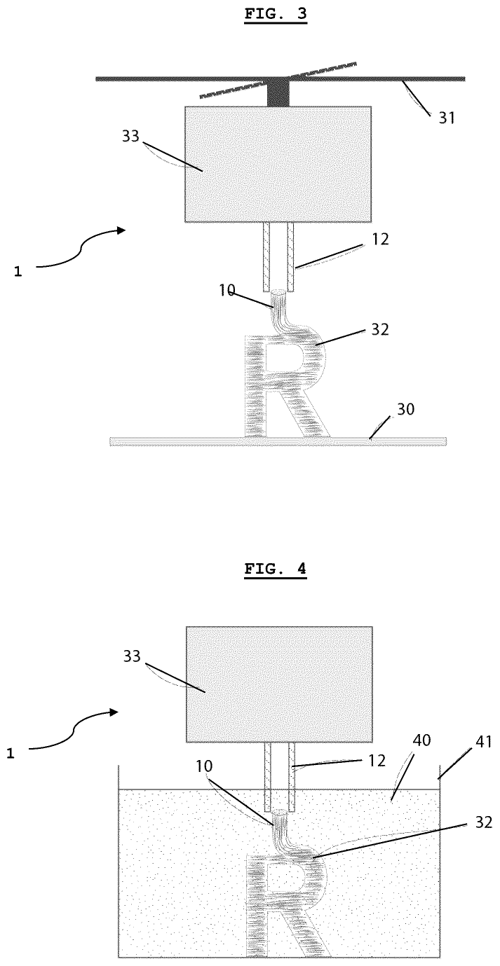

In another embodiment of the present invention, the system 1 comprising said print head 33 may furthermore comprise a print bed 30 and a means 31 for controlling the relative spatial position of said extrusion unit, preferably nozzle 12, and said print bed 30. FIG. 3 shows a non-limiting example of an embodiment with a print bed 30 and a means 31 for controlling the relative spatial position of the extrusion unit, preferably nozzle 12 and the print bed 30.

Said means 31 (schematically shown in FIG. 3) may be any component that can provide for a movement of a system 1 connected thereto. For example, it may comprise a rod that is connected at its terminal end with said print head 33. Said rod may be moved into and out of a cylindrical unit, for example by means of a motor, such as an electric motor, or pneumatically, thus moving the print head 33 in a vertical direction. If in addition or alternatively a horizontal movement of the print head 33 is desired, respective further or alternative known components may be used, for example a horizontally arranged rod similar to the rod described above.

Said print bed 30 may be any support onto which said filament 10 may be deposited. For example, it may be a substrate made of a polymeric material such as polyethylene or polypropylene.

The embodiment described in FIG. 3 is of particular interest when fabricating a one-dimensional, two-dimensional or three-dimensional structure 32 made of highly-aligned micro-filaments 10 by actuating the relative spatial position of the print head 33 according to FIG. 1 or 2 relatively to the print bed 30 to deposit the formed filament 10 made of highly-aligned micro-filaments 11 concurrently to its extrusion through the extrusion unit, preferably nozzle 12. For example, the print head 33 may be moved in horizontal and vertical direction such that photoresponsive material 13 is extruded in such a manner that it forms a structure having the form of the character “R”.

As described above, the system 1 comprises a means 16 for controlling the intensity of said one or more light sources 14, 21. Controlling the intensity of the light sources 14, 21 allows controlling the degree of crosslinking of the photoresponsive material.

According to another preferred embodiment of the present invention, the system 1 comprises a means for controlling the spatial coherence of said one or more light sources 14, 21. An optical modulation instability is more easily generated in a photoresponsive material 13 when using a spatially coherent light source 14, 21. In addition, the size of the micro-filaments 11 formed by an optical modulation instability in a photoresponsive material 13 depends on the spatial coherence of the light source 14, 21 that led to the optical modulation instability, with higher spatial coherence leading to smaller highly-aligned micro-filaments 11. Controlling the spatial coherence of the light source 14, 21 concurrently to the extrusion of the photoresponsive material 13 thus enables tuning the size of the formed micro-filaments 11 within the extruded filament 10.

Controlling the spatial coherence of a light source 14, 21 can be achieved for example, by placing a rotating ground glass diffuser in the path of the light beam emitted by the light source 14, 21.

When using a laser light source, the spatial coherence of the emitted light beam can be controlled by tuning the intra-cavity laser modes with a binary mask.

Another non-limiting example of a means of controlling the spatial coherence of a light source is to first select a light source with a low spatial coherence, such as an LED light source or a mercury lamp or a filament lamp, and to limit the emitting size of the light source with for instance a diaphragm. Limiting the emitting size of the light source to a small spatial extent will increase the degree of spatial coherence of the light source, thus leading to smaller generated micro-filaments or a more easily generated optical modulation instability within the photoresponsive material. On the other hand, increasing the emitting size of the light source will reduce the degree of spatial coherence of the said selected light source, thus yielding larger micro-filaments in the photoresponsive material and less ability of the light source to generate an optical modulation instability within the photoresponsive material.

It was found that a preferred embodiment of the system of the present invention comprises one or more partially spatially coherent light sources with a beam-parameter product less than 400 μm·rad, more preferably less than 100 μm·rad and most preferably less than 50 μm·rad.

According to another detailed embodiment the present invention is related to a method for fabricating a structure 32 having at least one dimension and made of highly aligned structural micro-components 11, the method comprises the steps of:
a) Providing a photoresponsive material 13 capable of altering its material phase upon illumination by light of one or more wavelengths;
b) Providing one or more light sources 14 capable of emitting light of one or more wavelengths, at least one of said one or more light sources 14 having the capability to generate an optical modulation instability in said photoresponsive material;

c) Providing a nozzle 12 to deposit said photoresponsive material 13;

d) Extruding said photoresponsive material 13 through said nozzle 12 and concurrently irradiating said photoresponsive material with said one or more light sources 14, thereby creating an optical modulation instability in said extruded photoresponsive material, thereby forming a micro-patterned filament 10;

e) Concurrently depositing said formed micro-patterned filament 10 to form the fabricated structure 32, thereby fabricating said structure 32 made of highly aligned structural micro-filaments 10.

In a further preferred embodiment of the present invention, the system 1 comprises a container in which the photoresponsive material is deposited. A non-limiting example of an embodiment comprising a container is shown in FIG. 4. In this embodiment, the print head 33 according to FIG. 1 or 2 of the system 1 for micro-patterned filament generation is arranged to deposit the formed micro-patterned filament 10 within a support material, preferably a Bingham plastic support material 40 contained in a container 41 to perform embedded printing. In embedded printing, a structure material such as the filament 10 is deposited within a support material 40 to form a fabricated structure 32. The support material 40 is then removed to release the formed fabricated structure 32.

The support material 40 preferably has special properties: it is stationary for applied stress levels below a threshold stress, thus enabling it to support the deposited structure material such as the filament 10, preventing any deformation of the fabricated structure 32. The support material 40 flows at stress levels above the threshold stress levels, hence allowing for the motion of an extrusion unit, preferably a nozzle 12, within the support material 40. Embedded printing is of particular interest for forming structures made of soft structure material, which is often the case in biofabrication applications.

Examples of appropriate support materials for embedded printing are Bingham plastic materials such as hydrogels or micronized gelatin particulates. A Bingham plastic is a viscoplastic material that behaves as a rigid body at low stresses but flows as a viscous fluid at high stress. Thermo-reversible materials such as gelatin are relevant support materials for embedded printing since they are in a gel state at temperatures below a threshold temperature and in a liquid state above the threshold temperature, which allows releasing the fabricated structure 32 by warming up the container 41 and subsequently the support material 40.

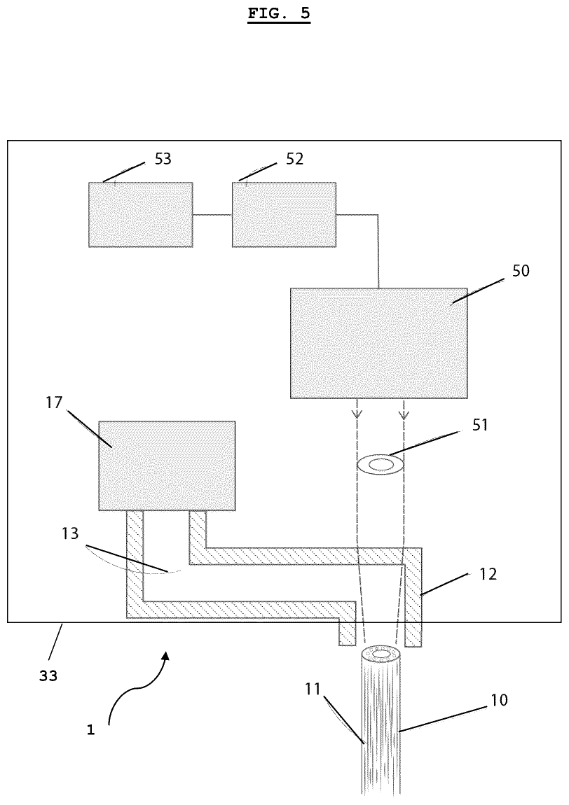

In a further preferred embodiment of the present invention, described in FIG. 5, a print head is provided for producing highly-aligned micro-filaments of tunable patterned geometry. In detail, a system 1 for the fabrication of an arbitrary length of a filament 10 made of highly aligned micro-filaments 11 with tunable patterned geometry comprises:
An extrusion unit, preferably a nozzle 12, to extrude a photoresponsive material 13;
A print head 33 comprising a light source in the form of a projection unit 50 capable of emitting spatial patterns 51 of light with one or more wavelengths of light, wherein the projection unit 50 is capable of generating an optical modulation instability in the photoresponsive material 13;
Whereby the projection unit 50 is arranged in the system such as to irradiate the photoresponsive material 13 when being in the extrusion unit, preferably a nozzle 12;
A means 52 for controlling the projection unit 50, said means being configured to cause emission of light from at least one of said light sources that is capable of generating an optical modulation instability in said photoresponsive material;
A means 53 for computing the spatial patterns of light 51;
A means 17 for controlling the extrusion of the photoresponsive material 13 through the extrusion unit, preferably nozzle 12.

The projection unit 50 is a device that may generate spatial patterns of light 51. The projection unit 50 may for example include a directly modulable light source such as an LED array, or it may include a light source with a fixed spatial profile (such as a laser or an LED) combined with a spatial light modulator. The spatial light modulator may consist of galvanometer-scanners, a liquid crystal spatial light modulator, or a digital micromirror device (DMD). The generated patterns of light may be zero-dimensional (spots), one-dimensional (lines), two-dimensional (images), or three-dimensional (holograms). One skilled in the art will understand that the projection unit 50 may incorporate additional optical elements, for example a cylindrical lens to correct for the distortion caused by a cylindrical container, or relay lenses to accurately project the light patterns inside the extrusion unit 12.

Said means 52 for controlling the projection unit 50 can be any known means that is conventionally used for this purpose, and is configured to cause emission of light from at least one of said light sources that is capable of generating an optical modulation instability in said photoresponsive material. For example, a conventional processing unit such as a computer may be used, which is respectively configured, for example, by the provision of a respective software.

Said means 53 for computing the spatial patterns of light 51 can be any known means that is conventionally used for this purpose. For example, a conventional processing unit such as a computer may be used.

It is also possible to provide more than one projection units 50 which may irradiate the photoresponsive material 13 with spatial patterns of light 51 from different angles.

Said means 53 is used for computing a sequence of projections for at least one of said light sources (50), said sequence of projections describing the micro-patterned filaments (11) of said structure (32).

The embodiment illustrated in FIG. 5 is of interest for producing for example a hollow filament geometry 10 made of highly-aligned micro-filaments 11.

Thus, according to this preferred embodiment a method of forming an arbitrary length of a filament 10 made of highly aligned micro-filaments 11 of tunable geometry is provided, wherein the method comprises the steps of:
a) Providing a photoresponsive material 13 capable of altering its material phase upon illumination by one or more wavelengths of light;
b) Providing a projection unit 50 capable of emitting spatial patterns of light 51 with one or more wavelengths of light, said projection unit 50 having the capability to generate an optical modulation instability in said photoresponsive material 13;
c) Providing an extrusion unit, preferably a nozzle 12 to deposit said photoresponsive material 13;
d) Computing a sequence of projections for the projection unit 50, said sequence of projection describing the micro-structural filaments 11 of said filament 10;
e) Defining a sequence of light patterns 51 using said sequence of projections;
f) Extruding the photoresponsive material 13 through the extrusion unit, preferably nozzle 12 and concurrently irradiating the photoresponsive material 13 with said sequence of light patterns 51 from said projection unit 50, thereby creating patterned optical modulation instability in said extruded photoresponsive material 13, thereby forming a tunable micro-patterned filament 10.

In another preferred embodiment, the system of the present invention comprises one or more projection units 50 capable of emitting spatial patterns of light with one or more wavelengths of light, wherein at least one of said one or more projection units 50 is capable of generating an optical modulation instability in said photoresponsive material. This embodiment is of particular interest when using a photoresponsive material 13 as described above that changes its material phase only upon the concurrent irradiation by light of two different wavelengths and does not change its material phase when irradiated by light of only one of the first or second wavelength. It is particularly advantageous to cure the photoresponsive material 13 just after the extrusion unit, preferably nozzle, to prevent any clogging of the system 1 by the formed micro-patterned filament 10.

In another preferred embodiment of the present invention, said one or more projection units 50 comprises a light source capable of emitting light of one or more wavelengths and at least one of a spatial light modulator, a digital micromirror device, a galvanometer-scanner, an acousto-optic deflector, a lens, a multimode fiber or a bundle of multimode fibers, as described above.

In a preferred embodiment of the present invention, the light source is selected from the group consisting of a laser diode, a diode-pumped solid-state laser, an OLED, a LED, and a combination of multiple LEDs.

According to another detailed embodiment of the invention, a method for fabricating a structure 32 having at least one dimension and made of highly aligned structural micro-components 11 comprises the steps of:
a) Providing a photoresponsive material 13 capable of altering its material phase upon illumination by light of one or more wavelengths;
b) Providing one or more projections units 50 capable of emitting spatial patterns of light 51 with one or more wavelengths, at least one of said projection units of spatial light patterns having the capability to generate an optical modulation instability in said photoresponsive material 13;
c) Providing a nozzle 12 to deposit said photoresponsive material 13;
d) Optionally providing a support material 41 for embedded printing of said structure 32, wherein said support material is a Bingham plastic material;
e) Computing a sequence of projections for at least one of said one or more projection units, said sequence of projections describing the micro-structural components of said structure;
f) Defining a sequence of light patterns 51 using said sequences of projections;
g) Extruding said photoresponsive material 13 through said nozzle 12 and concurrently irradiating said photoresponsive material 13 with said sequences of light patterns from said one or more projection units 50, thereby creating a patterned optical modulation instability in said extruded photoresponsive material, thereby forming a micro-patterned filament 10;
h) Concurrently depositing the formed micro-patterned filament 10 to form the fabricated structure, optionally performing said deposition in said support material, thereby fabricating said structure 32 made of highly aligned structural micro-components.

In another preferred embodiment of the present invention, the fabricated structure is made of multiple different photoresponsive materials, which can bring advanced functionalities to the fabricated structure. In said preferred embodiment, the present invention thus adds further steps to the method described hereabove in order to fabricate a multi-material structure having at least one dimension and made of highly-aligned structural micro-components 11, namely:
Providing another photoresponsive material 13 capable of altering its material phase upon illumination by one or more wavelengths of light;
Repeating steps (a) to (b) of the method described above with said other photoresponsive material until said multi-material structure is produced.

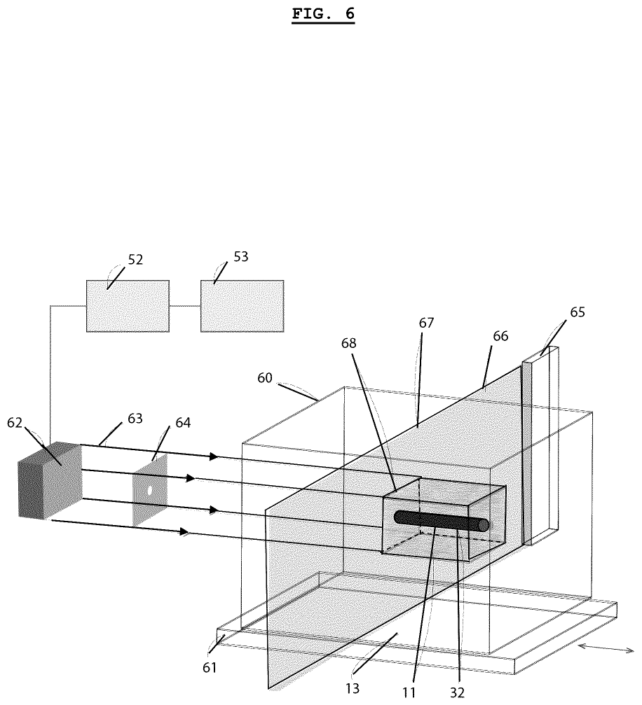

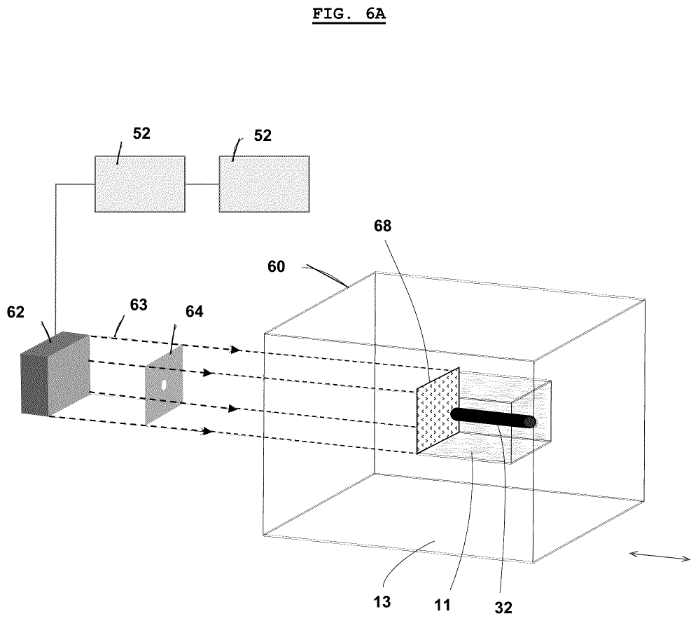

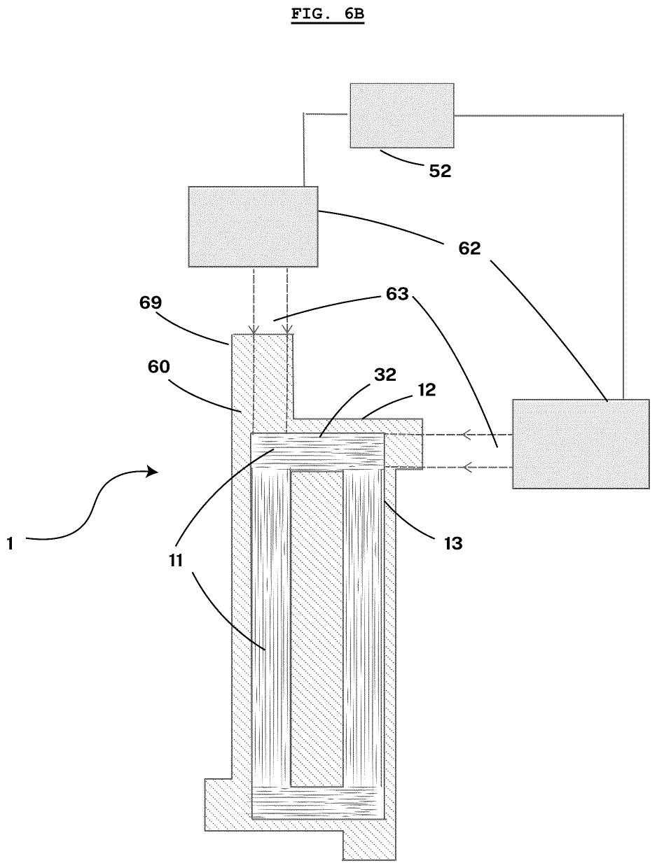

In a further preferred embodiment, a system for the fabrication of a structure 32 having at least one dimension and made of highly aligned structural micro-components 11 is provided and described in FIG. 6, wherein the system comprises:
An optically transparent vessel 60 in which a photoresponsive material 13 may be provided;
One or more preferably at least two, projection units 62, 65 capable of emitting spatial patterns of light 64 with light of one or more wavelengths into said optically transparent vessel 60, wherein at least one of said one or more projection units is configured to emit light that is capable of generating an optical modulation instability in said photoresponsive material 13;
A means 52 for controlling said one or more projection units 62, 65, said means 52 being configured to cause emission of light from at least one of said projection units 62, 65 that is capable of generating an optical modulation instability in said photoresponsive material 13;
A means 53 for computing said spatial patterns of light 64;
Optionally a means 61 for controlling the relative spatial position of said optically transparent vessel 60 and said one or more projection units 62, 65.

In a preferred embodiment of the invention, at least one of said one or more projection units 62, 65 is configured to irradiate said optically transparent vessel 60 with a light sheet 67. According to the present invention, a light sheet is a beam of light having the form of a sheet, i.e. having a thin rectangular shape.

According to the present invention, “an optically transparent vessel” is a vessel of any suitable shape (e.g. cuboid or cylindrical) whose walls are transparent, i.e. they do not absorb light in the visible range of the electromagnetic spectrum. Another example of optically transparent vessel is a transparent mold. As used herein, a mold is a body having a hollow inner space that corresponds to the shape of the article or structure to be formed.

The system described here and shown in FIG. 6 can be used for fabricating a structure 32 made of highly-aligned structural micro-filaments 11. In the embodiment of FIG. 6, a first projection unit 62 emitting light at a first wavelength 63 is arranged to irradiate with spatial patterns of light 64 the photoresponsive material 13 contained within the optically transparent vessel 60. This first projection unit 62 is capable of generating an optical modulation instability in the photoresponsive material 13. A second projection unit 65 emitting light at a second wavelength 66 is arranged to irradiate the photoresponsive material 13 with a light sheet 67 whose propagation direction is orthogonal to the propagation direction of the spatial patterns of light 64. A sequence of projections describing the structure 32 to be fabricated is computed by the means 53 and used to define the sequence of spatial light patterns 64. The photoresponsive material 13 is sequentially irradiated with the sequence of spatial light patterns 64, according to the defined sequence. For each light pattern of the sequence 64, the second projection unit 65 irradiates a defined layer 68 of the photoresponsive material 13. The concurrent irradiation of the photoresponsive material 13 by the two light beams of the first and second projection unit 62, 65 cures the layer 68 while inducing an optical modulation instability. The cured layer 68 is thus made of highly-aligned micro-filaments 11. The optically transparent vessel 60 is then moved orthogonally to the light sheet 67 propagation direction, as indicated by the arrow in FIG. 6. The projection, curing and displacement steps are repeated according to the defined sequence 64 until the structure 32 is fabricated.

This embodiment is of particular interest for xolography, a dual colour technique using photoswitchable photoinitiators to induce local polymerization inside a confined monomer volume upon linear excitation by intersecting light beams of different wavelengths (see e.g. Regehly et al., Nature, Vol. 588 (2020), 620-624). Accordingly, in this embodiment use is made of a photoresponsive material that changes its material phase only upon the concurrent irradiation by two different wavelengths of light and does not change its material phase when irradiated by light of only one of the first or second wavelength. It is particularly advantageous to cure only a selected layer 68 of the photoresponsive material contained within an optical vessel 60.

Thus, according to a preferred embodiment of the invention, a method is provided to fabricate a structure 32 having at least one dimension and made of highly-aligned structural micro-components 11, the method comprises the steps of:
a) Providing an optically transparent vessel 60 containing a photoresponsive material 13 capable of altering its material phase upon illumination by light of one or more wavelengths;
b) Providing one or more, preferably at least two, projections units 62, 65 capable of emitting spatial patterns of light 64 with one or more wavelengths, at least one of said one or more projection units 62, 65 of spatial light patterns 64 having the capability to generate an optical modulation instability in said photoresponsive material 13;
c) Computing a sequence of projections for at least one said one or more projection units 62, 65 describing the different layers of the structure 32 to be fabricated;
d) Defining a sequence of light patterns 64 for at least one of said one or more projection units 62, 65 using said computed sequence of projections;
e) Irradiating said photoresponsive material 13 with each of said light patterns 64 according to the defined sequences, preferably simultaneously irradiating said photoresponsive material 13 with a light sheet 67 whose propagation direction is orthogonal to the propagation direction of the spatial patterns of light 64, thereby sequentially generating a layer 68 of the structure 32 with an optical modulation instability, thereby sequentially curing a layer 68 of said structure 32, said layer being composed of highly aligned micro-filaments 11 generated by said optical modulation instability, thereby fabricating said structure 32 made of highly aligned structural micro-filaments 11.

This method can also be supplemented with further steps to fabricate a multi-material structure 32 having at least one dimension and made of highly-aligned structural micro-filaments 11, wherein the method comprises the further steps:
Removing uncured parts of said photoresponsive material and immersing said fabricated structure into another photoresponsive material into said optically transparent vessel;
Repeating steps (c) to (f) of the above method until said multi-material structure is produced.

An alternative system described here and shown in FIG. 6A can be used for fabricating a structure 32 made of highly-aligned structural micro-filaments 11. In the embodiment of FIG. 6A, a projection unit 62 emitting light 63 is arranged to irradiate with spatial patterns of light 64 the photoresponsive material 13 contained within the optically transparent vessel 60. This projection unit 62 is capable of generating an optical modulation instability in the photoresponsive material 13. A sequence of projections describing the structure 32 to be fabricated is computed by the means 53 and used to define the sequence of spatial light patterns 64. The photoresponsive material 13 is sequentially irradiated with the sequence of spatial light patterns 64, according to the defined sequence. For each light pattern of the sequence 64, the projection unit 62 cures a layer 68 while inducing an optical modulation instability. The cured layer 68 is thus made of highly-aligned micro-filaments 11. The optically transparent vessel 60 is then moved along the axis of illumination, as indicated by the arrow in FIG. 6a. The projection, curing and displacement steps are repeated according to the defined sequence 64 until the micro-patterned structure 32 is fabricated.

The above-mentioned system is of particular interest for regenerative medicine or drug-screening applications since the optically transparent vessel 60 can be, but not limited to, a transparent well-plate, wherein the same small structure 32 is built in parallel in each of the wells of the plate.

In a further preferred embodiment, the optically transparent vessel is a transparent mold.

Hence, according to another preferred embodiment of the invention, a method is provided to fabricate a structure 32 having at least one dimension and made of highly-aligned structural micro-components 11, the method comprises the steps of:
a) Providing an optically transparent vessel 60 containing a photoresponsive material 13 capable of altering its material phase upon illumination by light of one or more wavelengths;
b) Providing a projection unit 62 capable of emitting spatial patterns of light 64 with one or more wavelengths, said projection unit 62 of spatial light patterns 64 having the capability to generate an optical modulation instability in said photoresponsive material 13;
c) Computing a sequence of projections for said projection unit 62 describing the different layers of the structure 32 to be fabricated;
d) Defining a sequence of light patterns 64 for said projection unit 62 using said computed sequence of projections;
e) Irradiating said photoresponsive material 13 with a light pattern 64 according to the defined sequence, thereby generating a layer 68 of the structure 32 with an optical modulation instability, thereby curing a layer 68 of said structure 32, said layer being composed of highly aligned micro-filaments 11 generated by said optical modulation instability,
f) Actuating the relative spatial position of said optically transparent vessel 60 and said projection unit 62,
g) Repeating steps (e) and (f) according to the defined sequence of light patterns, thereby fabricating said structure 32 made of highly-aligned structural micro-filaments 11.

This method can also be supplemented with further steps to fabricate a multi-material structure 32 having at least one dimension and made of highly-aligned structural micro-filaments 11, wherein the method comprises the further steps:
Removing uncured parts of said photoresponsive material and immersing said fabricated structure into another photoresponsive material into said optically transparent vessel;
Repeating steps (c) to (g) of the above method until said multi-material structure is produced.

In a further preferred embodiment, a system for the fabrication of a structure 32 having at least one dimension and made of highly aligned structural micro-components 11 is provided, wherein the system comprises:
An optically transparent vessel 60, preferably a transparent mold, in which a photoresponsive material 13 may be provided;
One or more, light sources 62, preferably projection units, capable of emitting light 63 of one or more wavelengths into said optically transparent vessel 60, preferably transparent mold, wherein at least one of said one or more light sources is configured to emit light that is capable of generating an optical modulation instability in said photoresponsive material 13;
A means 52 for controlling said one or light sources 62, said means 62 being configured to cause emission of light from at least one of said light sources 62 that is capable of generating an optical modulation instability in said photoresponsive material 13;
Optionally, a means for controlling the spatial coherence of said one or more projection units of said one or more light sources 62.
Optionally, said transparent vessel 60, preferably a transparent mold, comprises one or more waveguides 69 arranged to irradiate said photoresponsive material 13 from various directions, thereby leading to the generation of optical modulation instabilities according to various preferential directions.

The system described above is advantageous, for instance, for regenerative medicine purposes. An example of a use of said system comprises the steps of:
Scanning an organ or tissue of a patient,
Using this scan to form a transparent anatomical mold replicating the anatomy of the patient's organ or tissue,
Optionally, seeding a photoresponsive material 13 with cells, for instance, the patient's stem cells,
Providing said photoresponsive material 13 within said transparent anatomical mold,
Irradiating said photoresponsive material 13 with one or more light sources 62 capable of inducing an optical modulation instability within said photoresponsive material 13,
Thereby forming an organ or tissue 32 with highly-aligned micro-structural components.
Optionally removing said formed organ or tissue 32 from said transparent anatomical mold.

The system described above, comprising said transparent vessel with said optional one or more waveguides is of interest for the fabrication of a structure 32 having anisotropic properties stemming from highly-aligned structural micro-components along various preferential directions. Such anisotropic properties are essential to replicate the function of tissues of organs, which are themselves made of sub-structures with various directional properties e.g. tensile or vascular. FIG. 6B describes an example of an embodiment for the fabrication of a structure 32 with anisotropic properties. A photoresponsive material 13 is poured into an optically transparent mold 60 and irradiated by one or more light sources 62 emitting light 63 at one or more wavelengths through one or more waveguides 69, thereby creating a structure 32 made of highly aligned micro-filaments 11 in various directions. Said one or more light sources 62 being controlled by said means 52.

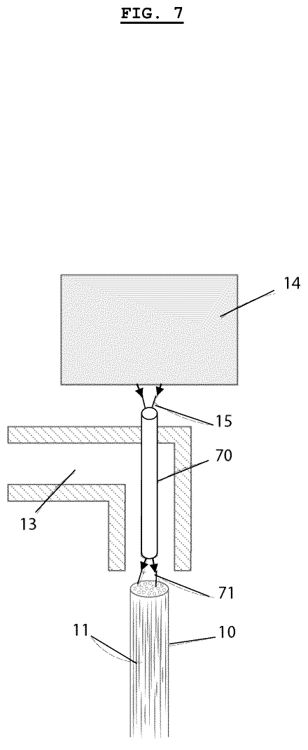

In a further embodiment of the invention, the system may comprise a means to guide light through said extrusion unit, preferably nozzle. Examples of guiding means include, but are not limited to, a multimode fiber, a bundle of multimode fiber, a lens or a prism. FIG. 7 shows an embodiment wherein light 15 from a light source 14 is guided towards a nozzle 12 through a multimode optical fiber 70. The irradiation of the photoresponsive material 13 extruded through the nozzle 12 by the guided light 71 yields an optical modulation instability and the formation of micro-filaments 11 within the extruded filament 10.

In a further embodiment of the invention, the system comprises a means for guiding spatial patterns of light from the one or more projection units towards an extrusion unit, preferably a nozzle. Examples of guiding means include, but are not limited to, a multimode fiber, a bundle of multimode fiber, a lens or a prism.

In a further embodiment of the invention, the system comprises a means for guiding spatial patterns of light from the one or more projection units towards the optically transparent vessel. Examples of guiding means include, but are not limited to, a multi-mode fiber, a bundle of multimode fiber, a lens or a prism.

In a preferred embodiment of the invention, said photoresponsive material 13 is seeded with cells. In other words, cells are provided in the photoresponsive material in the uncured state and thus are also present in the fabricated structure. This can be used in biofabrication applications wherein the biofabricated objects locally require highly aligned structures to guide cellular growth or function.

In a preferred embodiment, the micro-filaments' geometrical parameters are modulated through a physical parameter selected from the group consisting of:
the intensity of said one or more light sources;
the photoresponsive material flow rate through said nozzle;
or a combination thereof.

In a further preferred embodiment, the system further comprises any one of the following list:
a means to control the moist of said print bed, said printing container or said optically transparent vessel;
a means to control the temperature of said print bed, said printing container or said optically transparent vessel;
a means to control the carbon dioxide content of said print bed, said printing container or said optically transparent vessel;
or a combination thereof.

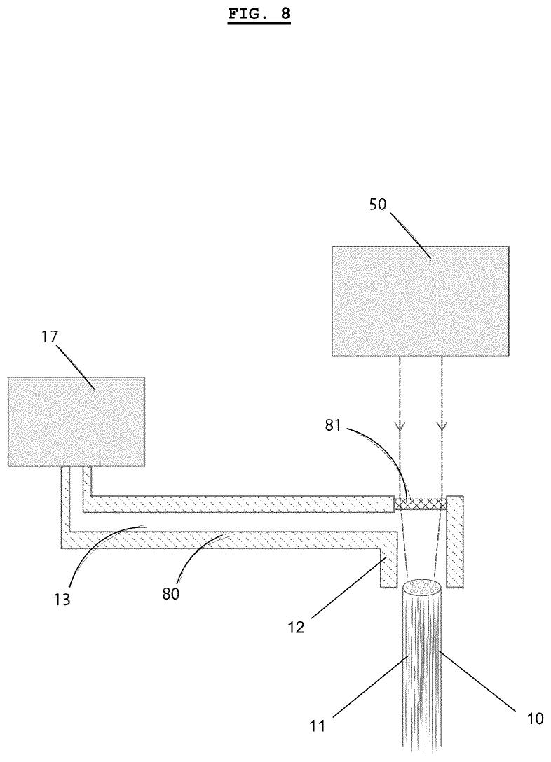

In a further preferred embodiment, the means 17 for controlling the extrusion provides a flow of photoresponsive material 13 through a microfluidic chip 80, with a projection unit 50 irradiating light through one location of the microfluidic chip 80, called projection window 81, as exemplified in FIG. 8.

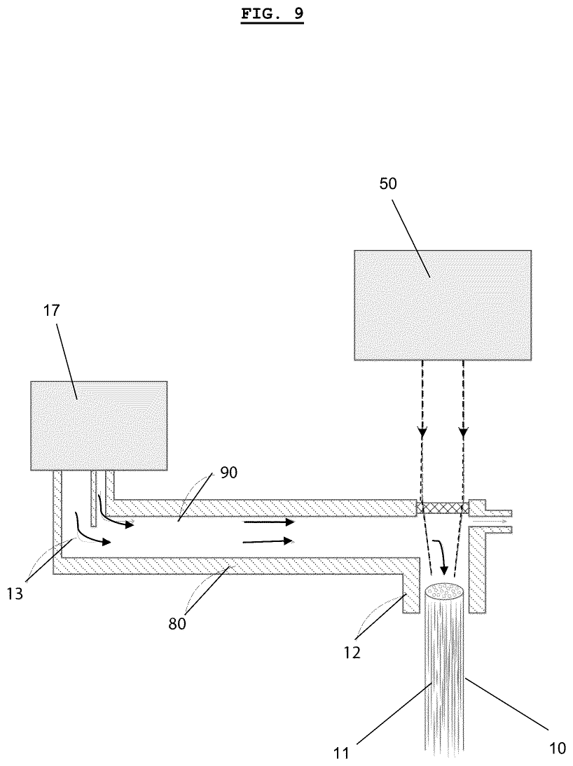

In a preferred embodiment, depicted in FIG. 9, said microfluidic chip 80 allows a sacrificial material 90, which can be any type of material that is not photoresponsive to light, for instance phosphate-buffered saline, to flow parallel to said photoresponsive material 13 with the intent to facilitate the extrusion of the cured filament 10 and to prevent any clogging of the nozzle 12.

In a preferred embodiment, said projection window 81 can be made from a material selected from the group consisting of:
an oxygen diffusing material, such as, but not limited to, Teflon AF 2400
a chemically treated glass preventing any material to attach to it
a transparent plastic material.

In a preferred embodiment, the means for controlling the extrusion 17 is compatible with a variety of microfluidic chips, each allowing a distinct flow geometry of one or more material.

In a preferred embodiment, said microfluidic chip 81 allows two or more materials to flow, thus enabling the fabrication of multi-material structures.

It is to be understood that the above-described embodiments can be combined with each other. For example, in each of the embodiments shown in the figures a projection unit or a normal light source may be used as a light source. Also, each of the embodiments shown in the figures any of the photoresponsive materials described herein may be used.

The present invention is not particularly limited with respect to the photoresponsive material to be used.

In a preferred embodiment of the present invention, the provided photoresponsive material comprises at least one component selected from the group consisting of:
a monomer;
a prepolymer;
one or more photo-initiators that interact with said light or another light source to selectively alter the phase of said photoresponsive material;
a chain extender;
a reactive diluent;
a filler;
a colorant, such as a pigment or dye;
or a combination of thereof.

In a further preferred embodiment, the phase of said photoresponsive material prior to said irradiation by said light source is that:
of a solid;
of a liquid;
of a gel.

In a further embodiment, said photo responsive material may contain:
micro-cellulose fibers;
particles;
cell supporting material;
biologically active components such as, but not limited to, growth factors for cell differentiation;
cells;
or a combination of thereof.

The present invention will be described hereinafter with respect to non-limiting examples.

### Example 1: Composition of the Photoresponsive Material for the Fabrication of a Structure With Highly-aligned Structural Micro-components

An example of a liquid photoresponsive material for the fabrication of a structure having at least one dimension and made of highly-aligned structural micro-filaments is given below:

| | |
|---|---|
| Gelatin methacryloyl | 10.000% w/v |
| Phosphate-buffered saline | 89.963% w/v |
| Phenyl (2, 4, 6- | 0.037% w/v |
| trimethylbenzoyl)phosphinate | |
| (LAP) | |

The different components were mixed on a hot plate at 40° C. with a magnetic stirrer at 200 rpm during 20 min prior to the fabrication process.

### Example 2: Composition of the Photoresponsive Material Sensitive to two Different Wavelengths of Light for the Fabrication of a Structure With Highly-aligned Structural Micro-components

An example of a liquid photoresponsive material that cures only upon concurrent irradiation by two different wavelengths of light but does not cure when irradiated with only one of said wavelengths of light is given in the table below. The term DCPI in the table below stands for dual-color photo-initiator, the dual-color photo-initiator can be selected from the group consisting of:
A spiropyran;
A spirooxazine;
A spiroimidazoquinoline-indoline;
A benzothiazole.

Such a photoresponsive material can be used for the fabrication of a structure having at least one dimension and made of highly-aligned structural micro-components in the embodiment comprising an optically transparent vessel as described in FIG. 6. The wavelengths of the first projection unit 62 can be, but not limited to, 565 nm. The wavelength of the light sheet beam 67 can be, but is not limited to, 375 nm.

| | |
|---|---|
| Pentaerythritol tetraacrylate (PETA) | 95 wt % |
| Triethanolamine | 4.988 wt % |
| Dual-color photo-initiator (DCPI) | 0.012 wt % |

The DCPI can be dissolved in ethyl-acetate or ethanol prior to adding it to the other components.

The different components were combined by mechanical stirring, followed by 10 min centrifugation at 4000 rpm for removal of residual air bubbles.

### Example 3: Composition of the Photoresponsive Material Sensitive to two Different Wavelengths of Light for the Fabrication of a Structure With Highly-aligned Structural Micro-components

An example of a liquid photoresponsive material that cures only upon concurrent irradiation by two different wavelengths of light but does not cure when irradiated with only one of said wavelengths of light is given in the table below. The term DCPI in the table below stands for dual-color photo-initiator, the dual-color photo-initiator can be selected from the group consisting of:
A spiropyran;
A spirooxazine;
A spiroimidazoquinoline-indoline;
A benzothiazole.

Such a photoresponsive material can be used for the fabrication of a structure having at least one dimension and made of highly-aligned structural micro-components in the embodiment comprising an optically transparent vessel as described in FIG. 6. The wavelengths of the first projection unit 62 can be, but not limited to, 565 nm. The wavelength of the light sheet beam 67 can be, but not limited to, 375 nm.

| | | |
|---|---|---|
| diurethane dimethacrylate (UDMA) | 98.988 | wt % |
| N-methyldiethanolamine | 1 | wt % |
| Dual-color photo-initiator (DCPI) | 0.012 | wt % |

The DCPI can be dissolved in ethyl-acetate or ethanol prior to adding it to the other components.

The different components were combined by mechanical stirring, followed by 10 min centrifugation at 4000 rpm for removal of residual air bubbles.

### Example 4: Composition of the Photoresponsive Material Sensitive to two Different Wavelengths of Light for the Fabrication of a Structure With Highly-aligned Structural Micro-components

An example of a liquid photoresponsive material that cures only upon concurrent irradiation by two different wavelengths of light but does not cure when irradiated with only one of said wavelengths of light is given in the table below. The term DCPI in the table below stands for dual-color photo-initiator, the dual-color photo-initiator can be selected from the group consisting of:
A spiropyran;
A spirooxazine;
A spiroimidazoquinoline-indoline;
A benzothiazole.

Such a photoresponsive material can be used for the fabrication of a structure having at least one dimension and made of highly-aligned structural micro-components in the embodiment comprising an optically transparent vessel as described in FIG. 6. The wavelengths of the first projection unit 62 can be, but not limited to, 565 nm. The wavelength of the light sheet beam 67 can be, but not limited to, 375 nm.

| | | |
|---|---|---|
| diurethane dimethacrylate (UDMA) | 97 | wt % |
| N-methyldiethanolamine | 0.5 | wt % |
| 2-hydroxyethylmethacrylate | 2.494 | wt % |
| Dual-color photo-initiator (DCPI) | 0.006 | wt % |

The DCPI can be dissolved in ethyl-acetate or ethanol prior to adding it to the other components.

The different components were combined by mechanical stirring, followed by 10 min centrifugation at 4000 rpm for removal of residual air bubbles.

### Example 5: Composition of the Photoresponsive Material Sensitive to two Different Wavelengths of Light for the Fabrication of a Structure With Highly-aligned Structural Micro-components

An example of a liquid photoresponsive material that cures only upon concurrent irradiation by two different wavelengths of light but does not cure when irradiated with only one of said wavelengths of light is given in the table below. The term DCPI in the table below stands for dual-color photo-initiator, the dual-color photo-initiator can be any one of the following list:
A spiropyran;
A spirooxazine;
A spiroimidazoquinoline-indoline;
A benzothiazole.

Such a photoresponsive material can be used for the fabrication of a structure having at least one dimension and made of highly-aligned structural micro-components in the embodiment comprising an optically transparent vessel as described in FIG. 6. The wavelengths of the first projection unit 62 can be, but not limited to, 565 nm. The wavelength of the light sheet beam 67 can be, but not limited to, 375 nm.

| | |
|---|---|
| acrylamide/bis-acrylamide | 90.9 wt % |
| (19/1, 40% in water) | |
| Triethanolamine | 9.09 wt % |
| Dual-color photo-initiator (DCPI) | 0.01 wt % |

The DCPI can be dissolved in ethyl-acetate or ethanol prior to adding it to the other components.

The different components were combined by mechanical stirring, followed by 10 min centrifugation at 4000 rpm for removal of residual air bubbles.

Example 6: Composition of the Photoresponsive Material Sensitive to two Different Wavelengths of Light for the Fabrication of a Structure With Highly-aligned Structural Micro-components

An example of a liquid photoresponsive material that cures only upon concurrent irradiation by two different wavelengths of light but does not cure when irradiated with only one of said wavelengths of light is given in the table below. The term DCPI in the table below stands for dual-color photo-initiator, the dual-color photo-initiator can be any one of the following list:
A spiropyran;
A spirooxazine;
A spiroimidazoquinoline-indoline;
A benzothiazole.

Such a photoresponsive material can be used for the fabrication of a structure having at least one dimension and made of highly-aligned structural micro-components in the embodiment comprising an optically transparent vessel as described in FIG. 6. The wavelengths of the first projection unit 62 can be, but not limited to, 565 nm. The wavelength of the light sheet beam 67 can be, but not limited to, 375 nm.

| | |
|---|---|
| Gelatin methacryloyl (10% in | 90.9 wt % |
| phosphate-buffered saline) | |
| Triethanolamine | 9.09 wt % |
| Dual-color photo-initiator (DCPI) | 0.01 wt % |

The DCPI can be dissolved in ethyl-acetate or ethanol prior to adding it to the other components.

The different components were combined by mechanical stirring, followed by 10 min centrifugation at 4000 rpm for removal of residual air bubbles.

### Experimental Results

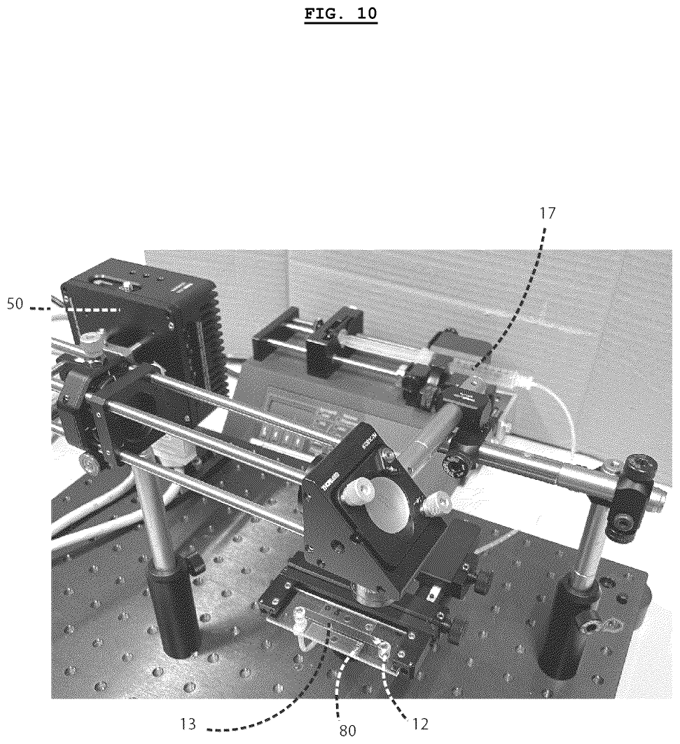

FIG. 10 depicts a device according to one embodiment of the present invention comprising a projection unit 50 irradiating light above the nozzle 12 of the microfluidic chip 80, in which the photoresponsive material 13 flows. Said photoresponsive material flow is controlled by a means for controlling the extrusion 17, in the non-limiting form of a pump.

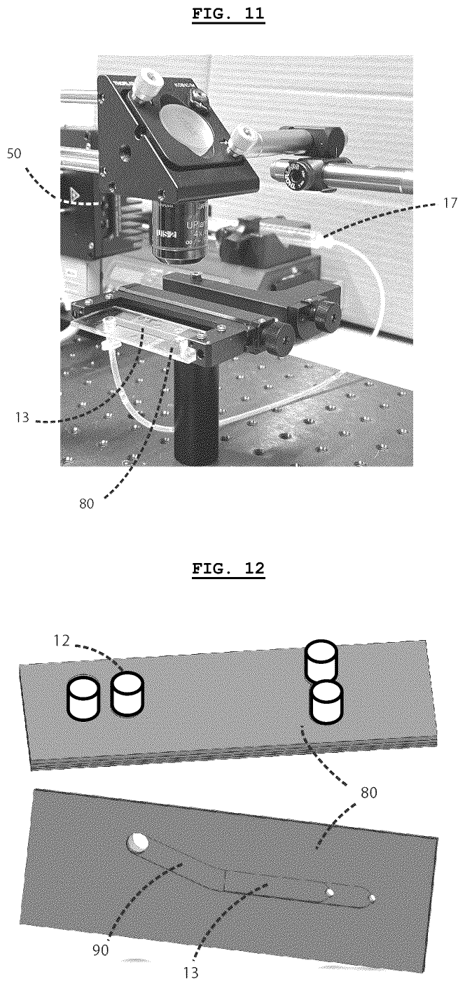

FIG. 11 provides a different view of the device of the embodiment presented in FIG. 10.

FIG. 12 depicts a non-limiting design of the microfluidic chip 80, comprising a sacrificial material 90 flowing collinearly with the photoresponsive material 13. Said microfluidic chip comprises the nozzle 12 from which the filament extrudes.

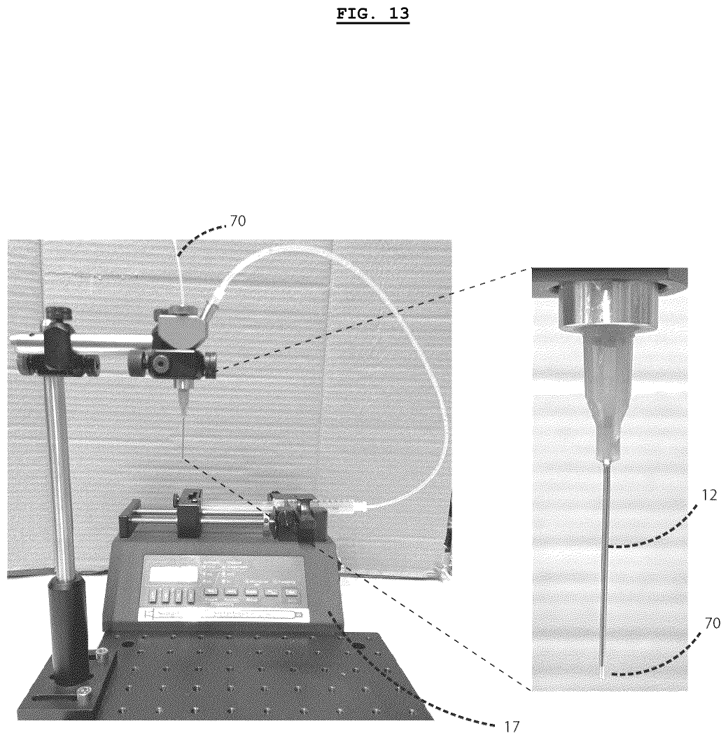

FIG. 13 depicts device according to another embodiment comprising a multimodal fiber 70 located inside the nozzle 12. A means for controlling the extrusion 17, here a syringe-pump, allows the photoresponsive material to flow collinearly and around said multimodal fiber.

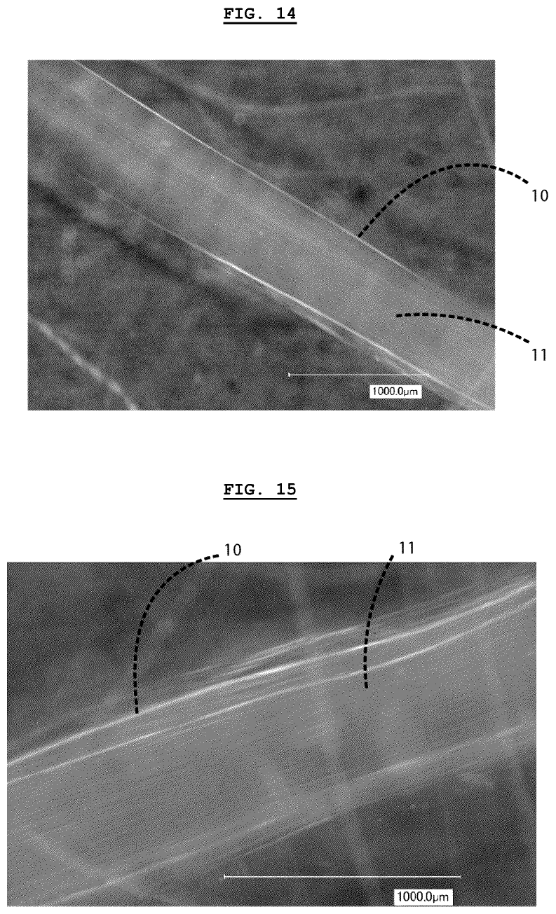

FIGS. 14 and 15 show experimental results of filaments 10 composed of highly aligned structural micro-components 11, obtained with aforementioned experimental embodiments.

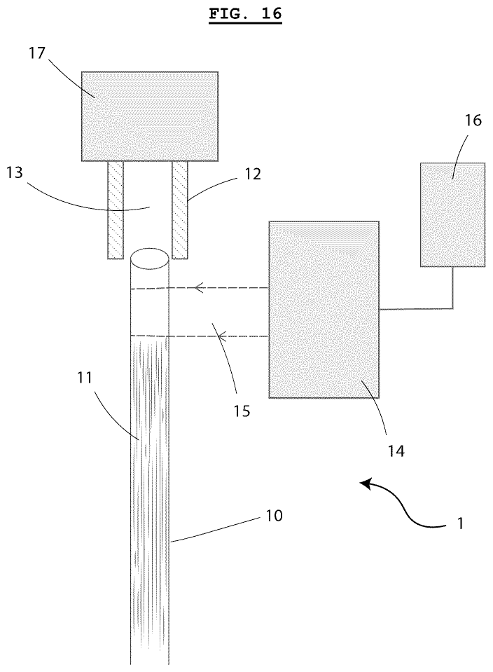

FIG. 16 shows another embodiment of the present invention, wherein said light source 14 of said system 1 is arranged to irradiate the photoresponsive material 13 after it has been extruded through said extrusion unit 12, preferably nozzle. The light source 14 is capable of generating an optical modulation instability in the photoresponsive material 13 and thus to generate a micro-patterned filament 10.

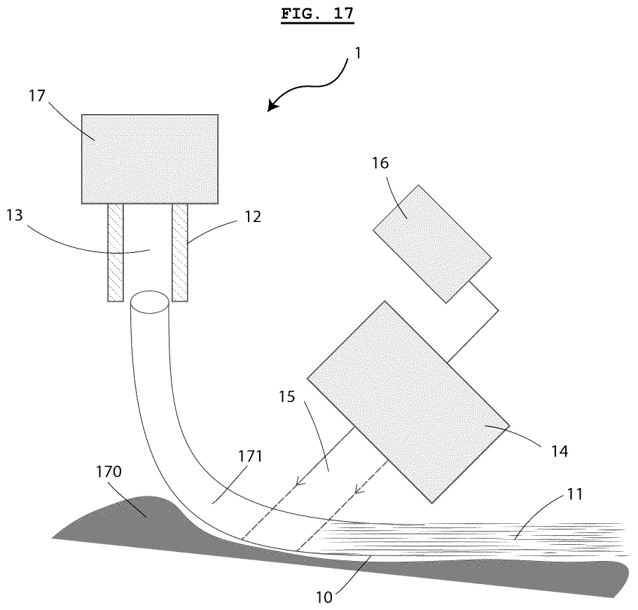

FIG. 17 shows another embodiment of the present invention, wherein said light source 14 of said system 1 is arranged to irradiate the photoresponsive material 13 after it has been extruded through said extrusion unit 12, preferably nozzle, and deposited for instance onto a print bed 30 or a body 170. This light source 14 is capable of generating an optical modulation instability in the deposited photoresponsive material 171 and thus to form a micro-patterned filament 10.

The embodiments described in FIGS. 16 and 17 are particularly advantageous to cure the photoresponsive material after the extrusion unit 12, preferably nozzle, to prevent any clogging of the system by the formed micro-patterned filament 10.

Moreover the embodiment described in FIG. 17 is particularly advantageous to form micro-patterned filaments 10 in situ onto a print bed or a body. For instance, the above embodiment could be used as follows: said photoresponsive material 13 is first ex-truded through said extrusion unit 12 and deposited onto the body of a patient and then irradiated with said light source 14 to form in situ a micro-patterned filament 10, for instance for regenerative medicine purposes.

1-15. (canceled)

16. A method for fabricating a structure having at least one dimension and made of highly aligned structural micro-filaments, the method comprising:
a) providing a photoresponsive material, said photoresponsive material being capable of altering its material phase upon illumination by light of one or more wavelengths, in an extrusion unit or in an optically transparent vessel;
b) irradiating said photoresponsive material with one or more light sources capable of emitting light of one or more wavelengths, at least one of said one or more light sources having a capability to generate an optical modulation instability in said photoresponsive material, thereby creating an optical modulation instability in said photoresponsive material, thereby forming a micro-patterned filament; and
c) forming from said formed micro-patterned filament said structure having at least one dimension and made of highly aligned structural micro-filaments.

17. The method according to claim 16, wherein said photoresponsive material is irradiated by two light sources that are arranged such that the paths of their emitted light are orthogonal with respect to each other.

18. The method according to claim 16, wherein said photoresponsive material is deposited onto or into a support, said support being selected from the group consisting of a print bed and a container.

19. The method according to claim 18, wherein said container comprises a support material.

20. The method according to claim 19, wherein after complete deposition of said photoresponsive material said support material is removed from said fabricated structure.

21. The method according to claim 16, wherein said one or more light sources emit spatial patterns of light with one or more wavelengths, at least one of said spatial light patterns having a capability to generate an optical modulation instability in said photoresponsive material, wherein said spatial light patterns have been generated by
computing a sequence of projections for at least one of said light sources, said sequence of projections describing the micro-patterned filaments of said structure, and
defining a sequence of light patterns using said sequences of projections.

22. The method according to claim 16, said method comprising the additional steps of:
d) Providing another photoresponsive material capable of altering its material phase upon irradiation with one or more wavelengths of light; and
e) Repeating steps (a) to (c) with said other photoresponsive material for producing a multi-material structure.

23. The method according to claim 16, wherein
said photoresponsive material capable of altering its material phase upon illumination by light of one or more wavelengths is contained in an optically transparent vessel, and
said optically transparent vessel of photoresponsive material is irradiated with spatial patterns of light with one or more wavelengths emitted by one or more projection units, wherein at least one of the one or more projection units has a capability to generate an optical modulation instability in said photoresponsive material,
said spatial patterns of light being generated by computing a sequence of projections for at least one of said one or more projection units describing different layers of the structure to be fabricated and defining a sequence of light patterns for at least one of said one or more projection units using said computed sequence of projections,
thereby sequentially generating a layer of the structure with an optical modulation instability, thereby sequentially curing a layer of said structure, said layer being composed of highly aligned micro-filaments generated by said optical modulation instability, thereby fabricating said structure made of highly aligned structural micro-filaments.

24. The method according to claim 23, comprising the following steps for producing a multi-material structure:
Removing uncured parts of said photoresponsive material and immersing said fabricated structure into another photoresponsive material in said optically transparent vessel; and
repeating the method of claim 23 until said multi-material structure is produced.

25. The method according to claim 23, wherein the relative position of said optically transparent vessel and at least one of said one or more projection units can be actuated and controlled concurrently to said irradiation of said optically transparent vessel.

26. The method according to claim 16, wherein the spatial coherence of at least one of said one or more light sources or said one or more projection units can be controlled and actuated concurrently to said irradiation, thereby modifying the size of said micro-patterns on said formed filament.

27. The method according to claim 16, wherein said photoresponsive material is seeded with cells.

28. A system for the fabrication of a structure having at least one dimension and made of highly aligned structural micro-components, the system comprising:
a) a unit in which a photoresponsive material can be irradiated, said unit being selected from the group consisting of an extrusion unit and an optically transparent vessel;
b) one or more light sources capable of emitting light of one or more wavelengths into said unit, wherein at least one of said one or more light sources is configured to emit light that is capable of generating an optical modulation instability in said photoresponsive material;
c) means for controlling said one or more light sources, said means being configured to cause emission of light from at least one of said light sources that is capable of generating an optical modulation instability in said photoresponsive material.

29. The system according to claim 28, wherein at least one of said one or more light sources is spatially coherent with a beam-parameter product less than 400 μm·rad.

30. The system according to claim 28, wherein at least one of said one or light sources is selected from the group consisting of a laser diode, a LED, an OLED, a diode-pumped solid-state laser, an incandescent filament and a combination of multiple LEDs.

31. The system according to claim 28 comprising a nozzle for controlling the extrusion of said photoresponsive material.

32. The system according to claim 28 comprising a support selected from the group consisting of a print bed and a container.

33. The system according to claim 28 comprising a nozzle for controlling the relative spatial position of said extrusion unit.

34. The system according to claim 28 comprising means for actuating and controlling the relative position of said optically transparent vessel and said one or more light sources.

35. The system according to claim 28 comprising a nozzle for guiding light from said one or more light sources towards said extrusion unit.
